(19) United States
(12) Patent Application Publication
(10) Pub. No.: US 2026/0168498 A1
(43) Pub. Date: Jun. 18, 2026

(54) FLOW SYSTEM

(71) Applicant: Sergio MOJARRO, Cypress, CA (US)

(72) Inventor: Sergio MOJARRO, Cypress, CA (US)

(21) Appl. No.: 18/986,565

(22) Filed: Dec. 18, 2024

(51) Int. Cl. F04D 13/06; A61M 60/117; A61M 60/216; A61M 60/411; A61M 60/806; F04D 29/046; F04D 29/42

(52) U.S. Cl. CPC F04D 13/068; A61M 60/117; A61M 60/216; A61M 60/411; A61M 60/806; F04D 29/046; F04D 29/426; F04D 29/4293

(57) ABSTRACT

A flow system has a centrifugal pump. The centrifugal pump has an impeller. The impeller has an impeller vane base. The centrifugal pump also has a pump head with a pump head upper section and a pump head lower section. A housing receives the centrifugal pump. A battery fits in a ballast frame. The reservoir has a liquid inlet port and the reservoir includes a pump inlet tube. The pump inlet tube is connected to a pump inlet of the centrifugal pump. The ballast frame is mounted to an underside of the housing underneath the centrifugal pump. The pump head lower section has a seal indent around a pump head lower section shaft opening. A shaft seal is mounted within the shaft indent of the pump head lower section.

### TECHNICAL FIELD

The present disclosure relates to a flow system, in particular a hemodynamic flow system.

### BACKGROUND

A variety of different pumps and centrifugal pumps have been used for hemodynamic flow.

### DISCUSSION OF RELATED ART

A variety of different flow systems have been discussed in United States patents. For example, in U.S. Pat. No. 4,589,822 entitled, “Centrifugal Blood Pump with Impeller,” by Earl W. Clausen and Lloyd C. Hubbard, published May 20, 1986, the inventors describe, “A centrifugal blood pump has an impeller with a hub, a blade support ring and alternating long and short blades. A tapered seal between a pump housing wall and a hub of an impeller which provides a fluid tight seal interface surrounding a shaft. The long blades of the impeller have rear edges which are closer to the wall than is the seal interface and inner edges which extend from the rear edges to the hub. This provides high flow in the vicinity of the seal interface to enhance heat dissipation from the seal interface”

For example, in U.S. Pat. No. 5,098,256 entitled, “Vicious Seal Blood Pump” by William A. Smith, published Mar. 24, 1992, the inventor describes, “A blood pump includes a housing having an inlet and outlet communicating with a pump chamber. A rotor received in the pump chamber is rotated by a drive motor. A drive shaft extending between the motor and rotor is sealed by a fluid seal. The housing includes an opening defining a clearance with the drive shaft on the order of one or two thousandth (0.002) of an inch. A viscous, biocompatible fluid is utilized as the seal fluid.”

For example, in U.S. Pat. No. 5,147,187 entitled, “Blood Pump and Extracorporeal Blood Circulating Apparatus,” by Kazuyuki Ito, Takeshi Aizawa, and Maktoto Tsuenda, the inventors describe, “An extracorporeal blood pump which comprises a pump housing having a pump chamber defined therein, and a rotary vane assembly accommodated within the pump chamber and including a substantially conical rotary pedestal having a base surface and a conical surface, a plurality of vanes each having radially inner and outer ends, and a driven shaft connected at one end with the base surface of the pedestal. The vanes are mounted on the conical surface so as to extend radially outwardly from an axis of rotation of the pedestal with the radially inner ends of the respective vanes being spaced a predetermined equal distance from an apex of the conical surface while substantially depicting a circle coaxial with the axis of rotation of the pedestal. Each neighboring members of the vanes are equally spaced from each other in a direction circumferentially of the pedestal. The base surface has a diameter of 30 to 55 mm enough to substantially cover a surface area of a bottom wall surface which partly defines the pump chamber and confronts the base surface of the pedestal, whereas each of the vanes is in the form of a generally rectangular straight plate extending at an angle of inclination within the range of 20 to 50 degrees relative to an imaginary line tangential to the circle delimited by the radially inner ends of the respective vanes. An extracorporeal blood circulatory device utilizing the blood pump of the type referred to above and comprising a control console accommodating a drive motor for the blood pump is also disclosed.”

For example, in U.S. Pat. No. 5,360,317 entitled, “Centrifugal Blood Pump,” by Earl. W. Clausen, Eden Prairie, and Lloyd Hubbard, published Nov. 1, 1994, the inventors describe, “A centrifugal pump for pumping biological fluids such as blood includes a housing which defines a pumping chamber. The pumping chamber ecloses an impeller mounted on a spindle. The impeller carries coupling mechanisms which couple with an external source of rotation to rotate the impeller. The spindle allows the impeller to rotate freely, but both ends of the spindle are constrained in the axial and lateral directions. The housing includes an inlet and an outlet.”

For example, in U.S. Pat. No. 5,458,459 entitled, “Centrifugal Blood Pump with Impeller Blades Forming a Spin Inducer,” by Earl. W. Clausen, Eden Prairie, and Lloyd Hubbard, published Oct. 17, 1995, the inventors describe, “A centrifugal pump for pumping biological fluids such as blood includes a housing which defines a pumping chamber. The pumping chamber encloses an impeller comprised of a spindle for rotation about a spindle axis and a plurality of blades positioned such that each inner blade end is positioned adjacent to the spindle. The plurality of inner blade ends forms a spin inducer which aids in decreasing hemolysis.”

For example, in U.S. Pat. No. 7,704,054 entitled, “Two-Stage Rotodynamic Blood Pump,” by David Horvath, Leonard A.R. Golding, and Alex Massiello, published Apr. 27, 2010, the inventors describe, “A pump (10) includes a housing, a stator (20) Supported in the housing, and a rotor assembly (30). The rotor assembly (30) includes a rotor (32) Supported in the housing for rotation relative to the stator (20) about an axis (12). The rotor assembly (30) also includes a first impeller (34) operatively coupled to a first axial end of the rotor (32) for rotation with the rotor about the axis (12). The rotor assembly further includes a second impeller (36) operatively coupled to a second axial end of the rotor (32), opposite the first axial end, for rotation with the rotor about the axis (12). The rotor assembly (30) is movable along the axis (12) relative to the housing to adjust hydraulic performance characteristics of the pump (10).”

For example, in United States publication number 200/0217962 entitled, “Medical Fluid Pump,” by Robert Childers and Don Busby, published Nov. 27, 2003, the inventors describe, “A method, System and apparatus for performing peritoneal dialysis are provided. To this end, in part, a pump for a dialysis System is provided. The pump includes a first chamber wall including a first aperture, a Second chamber wall including a Second aperture, a piston, at least a portion of which can move through the Second aperture, the piston including a third aperture, and first and second membranes disposed between the first and Second chambers walls. A Vacuum can be applied through the first aperture to pull the first membrane towards the first chamber wall and can be applied through the third aperture to pull the Second mem brane towards the piston. The piston can thereafter move the Second membrane.”

For example, in U.S. Pat. No. 7,850,593 entitled, “Fluid Pump,” by Douglas Vincent and Matthew J. Murphy, published Dec. 14, 2012, the inventors describe, “A pumping system 10, FIG. 1, provides a physiological pulsatile flow and includes controller 121, a pump drive head 50 coupled to a motor 12 and a fluid housing 52 having at least one port 60. The port 60 includes a ball valve retainer region 69, a valve seat 73, and an occluder ball 71 disposed in the ball valve retainer region 69. During operation, the motor 12 forces the fluid in and out the fluid housing 52 and causes the occluder ball 71 to move from a first position whereby the fluid cannot pass through the port 60, to a second position whereby the fluid moves annular to and generally around the occluder ball 71. This movement creates a slight flow reversal that “breaks up’ any blood clots that may form. The pumping system may be used as part of a cardiopulmonary bypass system, a ventricular assist device (VAD) and/or a heart pump.”

For example, in U.S. Pat. No. 11,300,119 entitled, “System For Driving A Pulsatile Fluid Pump,” by Douglas Vincent, Brian Bailey, Conrad Bzura, David Olney, Eric Smith, Jeffrey P. Naber, Juday Labonte, Kathleen Vincent, Matthew J. Murphy, and Patrick Shields, published Apr. 12, 2022, the inventors describe, “A pulsatile fluid pump system for driving a fluid pump assembly includes a reciprocating linear motor having a magnet and a coil, the magnet moving in relation to the coil, the coil having an electrical input. The pulsatile fluid pump system further includes a controller system having an electrical output coupled to the electrical input of the coil, and the controller system is configured to execute a waveform program defining an electrical waveform at the electrical output. The waveform program is configured to control operation of the linear motor by modification of a feature, selected from the group consisting of amplitude, frequency, shape, and combinations thereof, of the electrical waveform at the electrical output. The waveform program is further configured to accept a set of user-specifiable parameters defining the performance of the linear motor and to modify the electrical waveform in response to such parameters.”

For example, in U.S. Pat. No. 11,506,192 entitled, “Manual Driver Assembly for a Pulsatile Fluid Pump,” by David Olney, Douglas E. Vincent, Laura S. Cleminson, Lawrence Kuba, Michael Cole, and Roger Greeley, published Nov. 22, 2022, the inventors describe, “A pulsatile fluid pump apparatus includes an integral pump assembly employing a flexible diaphragm in a diaphragm assembly ; a pulsatile fluid pump system including a control housing, for removably receiving the integral pump assembly, the control housing and the integral pump assembly being configured for operation, in a first mode, so that the pulsatile fluid pump system reciprocally actuates the diaphragm assembly to cause pumping automatically ; and a manual driver assembly, wherein the integral pump assembly is configured, in a second mode, in the absence of actuation of the diaphragm assembly by the pulsatile fluid pump system, to removably receive the manual driver assembly to support manual actuation and operation of the diaphragm assembly.”

For example, in United States publication number 2022/0265993 entitled, “Pulsatile Fluid Pump System,” by Douglas E. Vincent, Brian Bailey, Conrad Bzura, David Butz, David Olney, Eric, Smith, George Koenig, James W. Poitras, Jeffrey P. Naber, Judy Labonte, Kathleen Vincent, Lawrence Kuba, Matthew J. Murphy, Patrick Shields, and Roger Greeley, published Aug. 25, 2022, the inventors describe, “A pulsatile fluid pump system includes a pump-valving assembly including a chamber and a diaphragm assembly coupled to the chamber and including a flexible diaphragm. The diaphragm assembly and the pump valving assembly are configured as an integral pump assembly. The system further includes a linear motor having a magnet and a coil, the magnet moving in relation to the coil, the coil having an electrical input. The system also includes a control housing rigidly coupled to the linear motor and a controller system having an electrical output coupled to the electrical input of the coil, the controller system defining an electrical wave form at the electrical output to cause desired operation of the diaphragm. The integral pump assembly is configured to be removably coupled to the control housing, and the diaphragm assembly of the integral pump assembly is configured to be removably coupled to the linear motor.”

For example, in United States publication number 2022/026994 entitled, “Pump-Valving Assembly for a Pulsatile Fluid Pump,” by Douglas E. Vincent, George Koenig, and Matthew J. Murphy, published Aug. 25, 2022, the inventors describe, “A pump-valving assembly for a pulsatile fluid pump includes a pumping chamber, an inlet port, and an outlet port. The pump-valving assembly further includes an inlet ball check-valve assembly, first and second tapered tracts disposed between the inlet port and the pumping chamber, an outlet ball check-valve assembly, and third and fourth tapered tracts disposed between the pumping chamber and outlet port. The first tapered tract expands in cross sectional area from the inlet port to the inlet ball check valve assembly, and the second tapered tract decreases in cross sectional area from the inlet ball check valve assembly to the chamber. The third tapered tract expands in cross sectional area from the chamber to the outlet ball check valve assembly and the fourth tapered tract decreases in cross sectional area from the outlet ball check valve assembly to the outlet port.”

For example, in United States publication number 2022/026994 entitled, “Diaphragm Assembly for a Pulsatile Fluid Pump,” by Douglas E. Vincent, George Koenig, James W. Poitras, and Matthew J. Murphy, published Aug. 25, 2022, the inventors describe, “A diaphragm assembly for a pulsatile fluid pump includes an edge-mounted flexible diaphragm, the diaphragm configured for operation cyclically between a diastole mode and asystole mode. The diaphragm assembly further includes a systolic distribution brace having an interior wall configured to cup a portion of the outside surface of the diaphragm, and a diastolic plate, embedded in the diaphragm, mechanically coupled to a portion of the inside surface of the diaphragm. In the course of the systole mode, force is applied across the maximum radial extent of the systolic distribution brace, so as to impart tension in the diaphragm around the periphery of the systolic distribution brace. In the course of the diastole mode, force is applied across the maximum radial extent of the diastolic plate, so as to impart tension in the diaphragm around the diastolic plate.”

### SUMMARY OF THE INVENTION

The present invention provides a flow system that is particularly adapted to hemodynamic flow which have medical use such as for aorta models and medical treatment. A flow system has a centrifugal pump. The centrifugal pump further includes an impeller. The impeller has an impeller vane base. The centrifugal pump further includes a pump head. The pump head further includes a pump head upper section and a pump head lower section. The pump head connects to a liquid outlet port. A housing receives the centrifugal pump. The battery fits in a ballast frame. The reservoir has a liquid inlet port and includes a pump inlet tube. The pump inlet tube is connected to a pump inlet of the centrifugal pump.

The ballast frame is mounted to an underside of the housing underneath the centrifugal pump. The pump head lower section further includes a seal indent around a pump head lower section shaft opening. A shaft seal is mounted within the shaft indent of the pump head lower section. The impeller has a first impeller vane, a second impeller vane, a third impeller vane, and a fourth impeller vane. The pump has a lower section with a motor housing bearing indent. The motor housing extends into the motor housing bearing indent. A shaft bearing is mounted within the motor housing main portion. The shaft bearing is mounted in the motor housing bearing indent.

The pump head lower section also has an annular alignment protrusion which extends into a motor housing bearing indent opening. The motor housing bearing indent opening is formed on the motor housing bearing indent. The aorta model is mounted to a first circulation tube connector and a second circulation tube connector. The first circulation tube connector is connected to a first circulation tube riser, and the second circulation tube connector is connected to a second circulation tube riser. The first circulation tube riser is connected to the liquid inlet port, and the second circulation tube riser is connected to the liquid outlet port. The platform supports the aorta model has a platform sidewall. The platform sidewall has illumination from an LED strip integrated thereto.

A switch assembly has a heater element socket for plugging in a heater. The heater hangs onto the reservoir rear wall. A control panel controls a pump controller. The control panel includes a first control knob for adjusting temperature, and a second control knob for adjusting flow rate. The control panel further includes a graphical display for displaying the flow rate and temperature.

The reservoir further includes a reservoir front wall and a reservoir rear wall. The reservoir rear wall is opposite the reservoir front wall. A reservoir step wall extends from the reservoir front wall to a reservoir lower wall. The reservoir lower wall extends between the reservoir rear wall and the reservoir step wall. The reservoir step wall has a reservoir step horizontal wall, and a reservoir step vertical wall. The reservoir step wall receives an upper corner edge of the ballast frame.

The pump head upper section connects to the pump head lower section at a pump head junction. The pump head junction has a pump head junction groove that receives a pump head seal. The pump head seal seals the pump head upper section to the pump head lower section. The pump head upper section does not touch the impeller. The impeller can have a webbed impeller base or a circular impeller base.

### BRIEF DESCRIPTION OF THE DRAWINGS

FIG. 1 is a perspective view of the present invention.

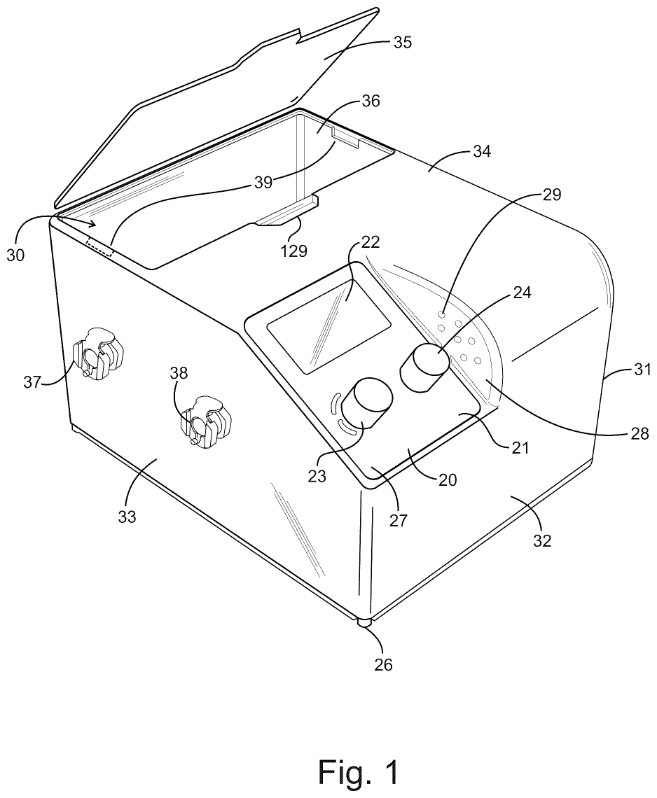

FIG. 2 is a cross-section view of the present invention.

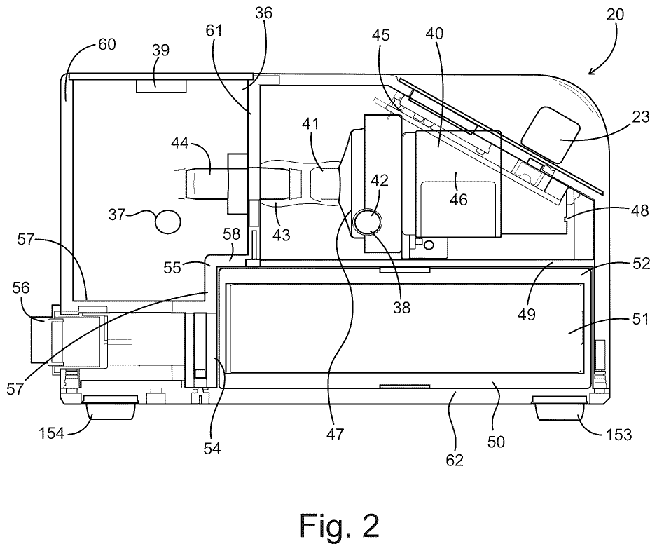

FIG. 3 is a cross-section of the pump head.

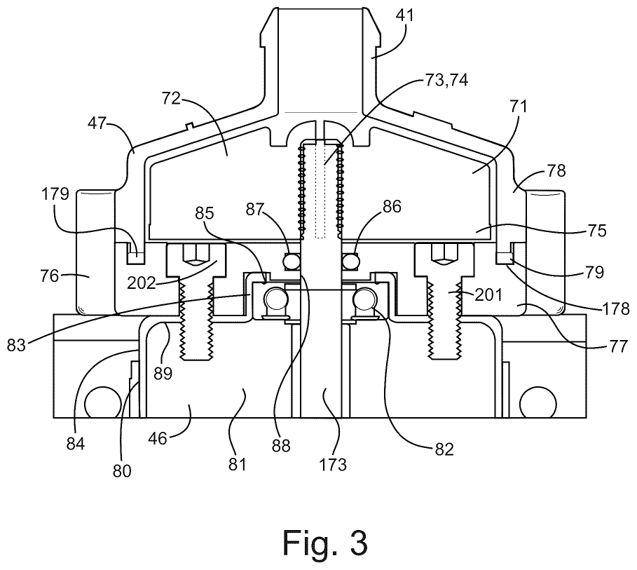

FIG. 4 is a cross-section of the present invention at a plane perpendicular to the cross-section view of FIG. 2.

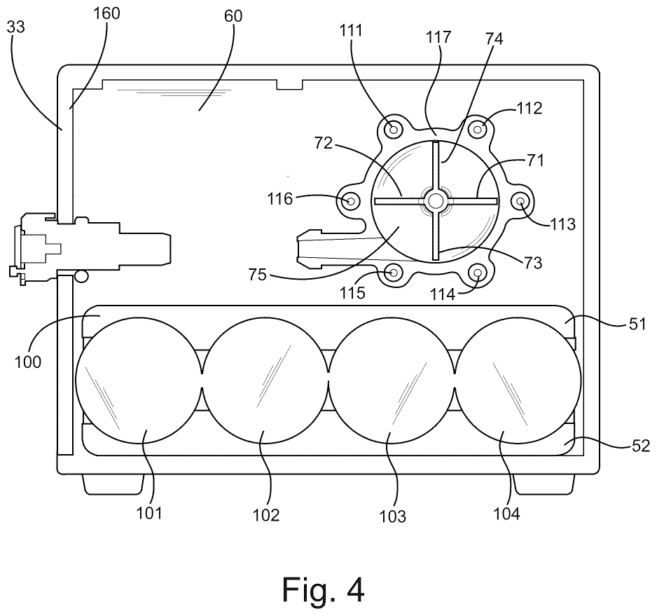

FIG. 5 is a perspective view of the present invention.

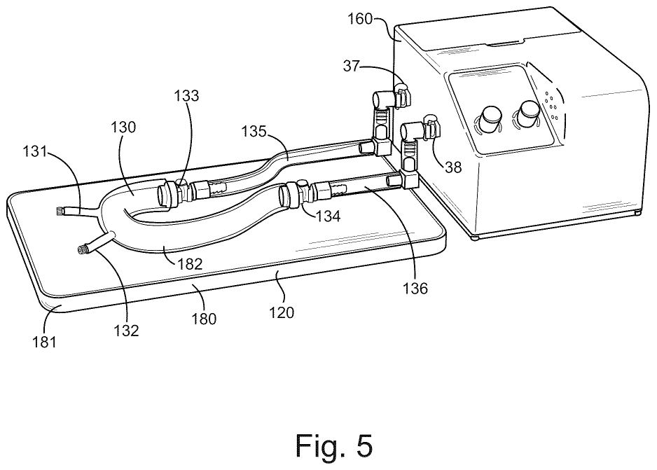

FIG. 6 is a close-up perspective view diagram of the pump head impeller showing the base webbing between the impeller vanes.

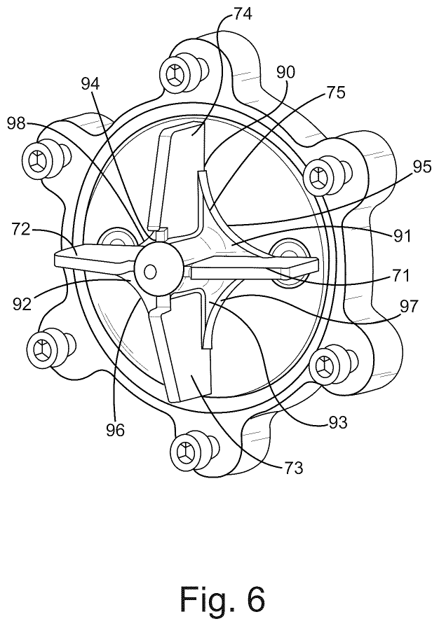

The following call out list of elements can be a useful guide in referencing the element numbers of the drawings.
20 Control
21 Control Panel
22 Display
23 First Control
24 Second Control
26 Housing Bottom
27 Housing Indent
28 Housing Indent Sidewall
29 Vent
30 Reservoir
31 Housing Right Surface
32 Housing Front Surface
33 Housing Left Side Surface
34 Housing Top Surface
35 Reservoir Lid
36 Reservoir Hollow
37 Liquid Inlet Port
38 Liquid Outlet Port
39 Lid Magnet
40 Pump
41 Pump Inlet
42 Pump Outlet
43 Pump Inlet Tube
44 Reservoir Bung
45 Pump Controller
46 Pump Motor
47 Pump Head
48 Pump Compartment
49 Pump Compartment Lower Wall
50 Ballast Compartment
51 Battery
52 Ballast Frame
54 Battery Compartment Rear Wall
55 Reservoir Step Wall
56 Switch
57 Reservoir Step Vertical Wall
58 Reservoir Step Horizonal Wall
59 Reservoir Lower Wall
60 Reservoir Rear Wall
61 Reservoir Front Wall
62 Ballast Compartment Opening
71 First Pump Impeller Vane
72 Second Pump Impeller Vane
73 Third Pump Impeller Vane
74 Fourth Impeller Vane
75 Pump Impeller Vane Base
77 Pump Head Lower Section
78 Pump Head Upper Section
79 Pump Head Junction
80 Pump Motor Housing
81 Pump Motor Windings
82 Shaft Bearing
83 Motor Housing Bearing Indent
84 Motor Housing Main Portion
85 Motor Housing Bearing Indent Opening
88 Pump Head Lower Section Shaft Opening
90 Webbing Attachment
91 First Base Webbing
92 Second Base Webbing
93 Third Base Webbing
94 Fourth Base Webbing
95 First Webbing Sidewall
96 Second Webbing Sidewall
97 Third Webbing Sidewall
98 Fourth Webbing Sidewall
100 Battery Cell Array
101 First Battery Cell
102 Second Battery Cell
103 Third Battery Cell
104 Fourth Battery Cell
111 First Pump Head Housing Connector
112 Second Head Housing Connector
113 Third Pump Head Housing Connector
114 Fourth Pump Head Housing Connector
115 Fifth Pump Head Housing Connector
116 Sixth Pump Head Housing Connector
117 Pump Head Housing
120 Platform
129 Lift Slot
130 Main Circulation Tube
131 First Circulation Tube Branch
132 Second Circulation Tube Branch
133 First Circulation Tube Connector
134 Second Circulation Tube Connector
135 First Circulation Tube Riser
136 Second Circulation Tube Riser
153 Front Foot
154 Rear Foot
160 Housing Shell
173 Pump Shaft
178 Pump Head Junction Slot
179 Pump Head Seal
180 Led Strip
181 Platform Sidewall
182 Aorta Model
201 First Retainer Bolt
202 Second Retainer Bolt
887 Annular Alignment Protrusion
888 Heater

### DETAILED DESCRIPTION OF THE EMBODIMENTS

As shown in the first figure, the present invention has a control 20 preferably mounted on a housing indent 27. The control 20 has a first control 23 and a second control 24. The first control 23 and the second control 24 can be formed as knobs. The control 20 has a control panel 21 which preferably has a display 22 for displaying a temperature and flow rate of the device. The display 22 can be a graphical display. The vent 29 can be formed on a housing indent sidewall 28 and the vent 29 may also have a speaker mounted within.
The liquid inlet port 37 and the liquid outlet port 38 are preferably mounted on a housing left side surface 33. The liquid inlet port 37 fluidly connects to the reservoir 30. The reservoir 30 is formed with a reservoir hollow 36 allowing a liquid store. A reservoir lid 35 of the reservoir 30 latches to the housing top surface 34 at a pair of lid magnets 39. The lid lift slot 129 is preferably formed on the housing top surface 34 allowing removal and replacement of the reservoir lid 35. The lid magnets 39 may allow placement of the reservoir lid 35 in a variety of different modes, such as a closed mode and an open mode.
The housing generally includes a housing right surface 31 opposite a housing left surface 33. The housing front surface 32 is opposite the housing rear surface. The housing bottom 26 is preferably open for accessing the battery structure.
As seen in the second figure, the reservoir 30 has a reservoir rear wall 60 opposite a reservoir front wall 61. The reservoir 30 also has a reservoir step wall 55 which includes a horizontal portion and a vertical portion. The reservoir step wall 55 has a reservoir step horizontal wall 58 and a reservoir step vertical wall 57. The reservoir step vertical wall 57 meets with the reservoir lower wall 59. The reservoir front wall 61 is preferably parallel to the reservoir step vertical wall 57. The reservoir step horizontal wall 58 is preferably parallel to the reservoir lower wall 59. The reservoir step vertical wall 57 is preferably parallel to the reservoir rear wall 60. Additionally, a heater 888 can hang on to the reservoir rear wall 60 and be controlled by the switch 56 mounted under the rear wall 60. The heater 888 can be a resistance heater that is controlled by the control panel.
The reservoir step wall 55 receives a portion of the ballast frame 52. The ballast frame receives the battery 51. The ballast frame can be compartmentalized for receiving multiple cells of the battery 51. The ballast frame provides weight and counterbalances the pump 40. The pump compartment lower wall 49 separates the pump 40 from the ballast frame 52. The pump compartment lower wall 49 is planar and extends above the ballast frame 52. The pump 40 can be mounted to the pump compartment lower wall 49.
The ballast compartment 50 receives the ballast frame 52. The battery compartment rear wall 54 extends downwardly from the reservoir step vertical wall 57. The housing front surface forms a housing front wall that bounds the ballast frame 52. The housing front surface is parallel to the battery compartment rear wall 54. The ballast compartment opening 62 may have a rectangular shape. The front foot 153 and the rear foot 154 are preferably elastomeric and dampen the vibration motion of the pump. The power switch 56 can be mounted on the rear wall of the housing. The rear wall of the housing is coextensive to the reservoir rear wall 60 and extends downwardly from the reservoir rear wall 60.
The pump 40 receives power from the battery 51 and receives control from the control 20. The control 20 includes a pump controller 45. The pump controller 45 is preferably mounted on a printed circuit board underneath the control panel 21. The pump compartment 48 receives the pump 40. The pump motor 46 powers the pump head 47 such that liquid passes from the liquid inlet port 37 to the reservoir 30, then through the reservoir bung 44 and into the pump inlet tube 43. The liquid then passes through the pump inlet 41 through the pump head 47 and exiting at the pump outlet 42. The pump outlet 42 is connected to the liquid outlet port 38 such that liquid enters through the liquid inlet port 37 and exits through the outlet port 38. The pump 40 is a horizontally mounted and mounted directly above the ballast frame 52. The pump 40 is thus indirectly but rigidly connected to the ballast frame 52.
The ballast compartment 50 receives the ballast frame 52. The battery compartment rear wall 54 extends downwardly from the reservoir step vertical wall 57. The housing front surface forms a housing front wall that bounds the ballast frame 52. The housing front surface is parallel to the battery compartment rear wall 54. The ballast compartment opening 62 may have a rectangular shape. The front foot 153 and the rear foot 154 are preferably elastomeric and dampen the vibration motion of the pump. The power switch 56 can be mounted on the rear wall of the housing. The rear wall of the housing is coextensive to the reservoir rear wall 60 and extends downwardly from the reservoir rear wall 60.
The pump 40 receives power from the battery 51 and receives control from the control 20. The control 20 includes a pump controller 45. The pump controller 45 is preferably mounted on a printed circuit board underneath the control panel 21. The pump compartment 48 receives the pump 40. The pump motor 46 powers the pump head 47 such that liquid passes from the liquid inlet port 37 to the reservoir 30, then through the reservoir bung 44 and into the pump inlet tube 43. The liquid then passes through the pump inlet 41 through the pump head 47 and exiting at the pump outlet 42. The pump outlet 42 is connected to the liquid outlet port 38 such that liquid enters through the liquid inlet port 37 and exits through the outlet port 38.
As seen in the third figure, the pump head 47 extends upwardly to a pump inlet 41. The pump head 47 includes a pump head upper section 78 and a pump head lower section 77. The impeller includes a first pump impeller vane 71, a second pump impeller vane 72, a third pump impeller vane 73, and a fourth pump impeller vane 74. The impeller is mounted to the pump shaft 173 outside of a shaft seal 86. The shaft seal 86 is preferably formed as an O-ring or elastomeric bushing retained within a seal indent 87. The seal indent 87 is preferably annular and notched into the pump head lower section 77.
The pump head upper section 78 meets with the pump head lower section 77 at a pump head junction 79. The pump head junction 79 further includes a pump head seal indent that receives a pump head seal 179. The pump head seal indent is the pump head junction groove 178 and is sized to receive the pump head seal 179. The pump head sidewall 76 can be annular and extended from the pump head 47.
The pump head 47 has an intent at a motor housing bearing indent 83 on the pump head lower section 77 where the pump head lower section 77 indents inwardly toward the seal indent 87. The pump head lower section 77 receives a first retainer bolt 201 and a second retainer bolt 202. The first retainer bolt 201 and the second retainer bolt 202 have a gap between them and the motor housing bearing indent 83 is formed between the first retainer bolt 201 and the second retainer bolt 202.
The motor housing bearing indent 83 receives a shaft bearing 82. The shaft bearing 82 is connected to the pump shaft 173 allowing the pump shaft 173 to rotate smoothly. The pump motor housing 80 has a motor housing bearing indent opening 85 formed at the motor housing bearing indent 83. The motor housing bearing indent opening 85 receives an annular alignment protrusion 887 that extends from the pump had lower section 77 into the motor housing bearing indent opening 85. The annular alignment protrusion 887 is a circular protrusion that engages the motor housing bearing indent opening 85 at a periphery of the annular locking protrusion 887. The annular alignment protrusion 887 aligns the pump head lower section 77 to the motor housing main portion 84. The annular alignment protrusion 887 preferably protrudes from the pump head lower section 77 as far as a thickness of the motor housing main portion 84. The motor housing main portion 84 is preferably a metal housing. The pump head lower section shaft opening 88 receives the pump shaft 173. The pump shaft 173 receives electrical force from the pump motor windings 81. The electrical force is controlled by the controller 20. The motor housing main portion upper side wall 89 is parallel to and preferably abuts the pump head lower section 77.
As shown in the fourth figure, a transverse cross-section view shows the battery 51 retained in the ballast frame 52. The battery doubles as ballast and therefore the ballast frame 52 receives the battery. The battery can be formed and include a first battery cell 101, a second battery cell 102, a third battery cell 103, and a fourth battery cell 104. Each of the battery cells are connected to form a single battery 51 as a battery cell array 100. The battery 51 can be inserted to be retained within the ballast frame 52. The ballast frame 52 is a battery holder and receives snugly within the housing shell 160. The housing shell 160 has an opening on its lower side for receiving the battery holder. The battery holder is preferably made of a flexible plastic frame to dampen vibrations from the impeller.
The pump head housing 117 which includes a pump head lower section 77 and the pump head upper section 78 has a number of connectors that connect the upper section to the lower section. For example, a pump head housing connector set may include a first pump head housing connector 111, a second pump head housing connector 112, a third pump head housing connector 113, a fourth pump head housing connector 114, a fifth pump head housing connector 115, and a sixth pump head housing connector 116. The pump head housing connectors can be formed as screws that pass through screw sockets at a periphery of the pump head housing.
The first impeller vane 71, the second impeller vane 72, the third impeller vane 73 and the fourth impeller vane 74 are at right angles to each other and mounted on a pump impeller vane base 75. The pump impeller vane base 75 can be circular and extend around the impeller vanes, or can be formed as a webbing between the impeller vanes.
As seen in the fifth figure, the housing show 160 houses the pump, reservoir and battery and control and can provide constant temperature and flow rate to an aorta model 182. The aorta model 182 preferably includes a main circulation tube 130 with a first circulation tube branch 131 and a second circulation tube branch 132. The aorta model 182 is preferably mounted to a platform 120 having an LED strip 180 mounted in a platform sidewall 181. The LED strip 180 can be translucent to provide indirect lighting through the platform surface and the platform sidewall 181 so as to illuminate the aorta model 182. The aorta model is preferably connected at a first circulation tube connector 133 and a second circulation tube connector 134. The first circulation tube connector 133 connects the main circulation tube 132 the first circulation tube riser 135. The second circulation tube connector 134 connects the main circulation tube 132 the second circulation tube riser 136. The first circulation tube riser 135 connects to the liquid inlet port 137 while the second circulation tube riser 136 connects to the liquid outlet port 38.
As seen in the sixth figure, the first pump impeller vane 71 and the fourth pump impeller vane 74 have a webbing attachment 90 between them formed at the pump impeller vane base 75. The webbing attachment 90 is a first base webbing 91 and has a first webbing sidewall 95. Similarly, the fourth pump impeller vane 74 and the second pump impeller vane 72 have a fourth base webbing 94 between them. The fourth base webbing 94 has a fourth webbing sidewall 98. The second pump impeller vane 72 and the third pump impeller vane 73 have a second base webbing 92 between them. The second base webbing 92 has a second webbing sidewall 96. The third pump impeller vane 73 and the first pump impeller vane 71 have a third base webbing 93 between them. The third base webbing 93 further includes a third webbing sidewall 97.
As used in this specification, the terms upper and lower are in reference to the elements as shown in the drawings, and not necessarily in actual use.

1. A flow system comprising:
a. a centrifugal pump, wherein the centrifugal pump further includes an impeller, wherein the impeller has an impeller vane base, wherein the centrifugal pump further includes a pump head, wherein the pump head further includes a pump head upper section and a pump head lower section, wherein the pump head connects to a liquid outlet port;
b. a housing receiving the centrifugal pump;
c. a battery, wherein the battery fits in a ballast frame; and
d. a reservoir, wherein the reservoir has a liquid inlet port, wherein the reservoir includes a pump inlet tube, wherein the pump inlet tube is connected to a pump inlet of the centrifugal pump.

2. The flow system of claim 1, wherein the ballast frame is mounted to an underside of the housing underneath the centrifugal pump.

3. The flow system of claim 1, wherein the pump head lower section further includes a seal indent around a pump head lower section shaft opening, wherein a shaft seal is mounted within the shaft indent of the pump head lower section.

4. The flow system of claim 1, wherein the impeller has a first impeller vane, a second impeller vane, a third impeller vane, and a fourth impeller vane.

5. The flow system of claim 1, wherein the pump has a lower section with a motor housing bearing indent, wherein the motor housing extends into the motor housing bearing indent, further including a shaft bearing mounted within the motor housing main portion, wherein the shaft bearing is mounted in the motor housing bearing indent.

6. The flow system of claim 5, wherein the pump head lower section further includes an annular alignment protrusion which extends into a motor housing bearing indent opening, wherein the motor housing bearing indent opening is formed on the motor housing bearing indent.

7. The flow system of claim 1, further including an aorta model, wherein the aorta model is mounted to a first circulation tube connector and a second circulation tube connector, wherein the first circulation tube connector is connected to a first circulation tube riser, and wherein the second circulation tube connector is connected to a second circulation tube riser, wherein the first circulation tube riser is connected to the liquid inlet port, wherein the second circulation tube riser is connected to the liquid outlet port.

8. The flow system of claim 7, further including a platform, wherein the platform supports the aorta model, wherein the platform has a platform sidewall, wherein the platform sidewall has illumination from an LED strip integrated thereto.

9. The flow system of claim 1, wherein the pump head upper section connects to the pump head lower section at a pump head junction, wherein the pump head junction has a pump head junction groove that receives a pump head seal, wherein the pump head seal seals the pump head upper section to the pump head lower section, wherein the pump head upper section does not touch the impeller.

10. A flow system comprising:
a. a centrifugal pump, wherein the centrifugal pump further includes an impeller, wherein the impeller has an impeller vane base, wherein the centrifugal pump further includes a pump head, wherein the pump head further includes a pump head upper section and a pump head lower section, wherein the pump head connects to a liquid outlet port, wherein the pump head lower section further includes a seal indent around a pump head lower section shaft opening, wherein a shaft seal is mounted within the shaft indent of the pump head lower section, wherein the impeller has a first impeller vane, a second impeller vane, a third impeller vane, and a fourth impeller vane, wherein the pump has a lower section with a motor housing bearing indent, wherein the motor housing extends into the motor housing bearing indent, further including a shaft bearing mounted within the motor housing main portion, wherein the shaft bearing is mounted in the motor housing bearing indent;
b. a housing receiving the centrifugal pump;
c. a battery, wherein the battery fits in a ballast frame, wherein the ballast frame is mounted to an underside of the housing underneath the centrifugal pump; and
d. a reservoir, wherein the reservoir has a liquid inlet port, wherein the reservoir includes a pump inlet tube, wherein the pump inlet tube is connected to a pump inlet of the centrifugal pump.

11. The flow system of claim 9, wherein the pump head lower section further includes an annular alignment protrusion which extends into a motor housing bearing indent opening, wherein the motor housing bearing indent opening is formed on the motor housing bearing indent.

12. The flow system of claim 9, further including a control panel controlling a pump controller, wherein the control panel includes a first control knob for adjusting temperature, and a second control knob for adjusting flow rate, wherein the control panel further includes a graphical display for displaying the flow rate and temperature.

13. The flow system of claim 9, further including a switch assembly, wherein the switch assembly further includes a heater element socket for plugging in a heater, wherein the heater hangs onto the reservoir rear wall.

14. The flow system of claim 9, wherein the reservoir further includes a reservoir front wall and a reservoir rear wall, wherein the reservoir rear wall is opposite the reservoir front wall, further including a reservoir step wall, wherein the reservoir step wall extends from the reservoir front wall to a reservoir lower wall, wherein the reservoir lower wall extends between the reservoir rear wall and the reservoir step wall, wherein the reservoir step wall has a reservoir step horizontal wall, and a reservoir step vertical wall.

15. The flow system of claim 13, wherein the reservoir step wall receives an upper corner edge of the ballast frame.

16. The flow system of claim 13, wherein the pump head upper section connects to the pump head lower section at a pump head junction, wherein the pump head junction has a pump head junction groove that receives a pump head seal, wherein the pump head seal seals the pump head upper section to the pump head lower section, wherein the pump head upper section does not touch the impeller.

17. The flow system of claim 13, wherein the impeller has a circular impeller base.

18. The flow system of claim 13, wherein the impeller has a webbed impeller base, wherein the impeller base further includes a first base webbing having a first base webbing sidewall, wherein the impeller base further includes a second base webbing having a second base webbing sidewall, wherein the impeller base further includes a third base webbing having a third base webbing sidewall.
